(19) United States
(12) Patent Application Publication
(10) Pub. No.: US 2010/0247613 A1
(43) Pub. Date: Sep. 30, 2010

(54) Dressing Modified to Receive Medicine

(75) Inventor: Randel B. Holmes, Knoxville, TN (US)

Correspondence Address: PITTS AND BRITTIAN P C, P O BOX 51295, KNOXVILLE, TN, 37950-1295, US

(73) Assignee: WEBTEC Converting, LLC., Knoxville, TN (US)

(21) Appl. No.: 12/415,605

(22) Filed: Mar. 31, 2009

(51) Int. Cl. A61L 15/10; A61F 13/02

(52) U.S. Cl. 424/445; 602/57

(57) ABSTRACT

A laminated dressing defining an area adapted to receive and hold medicine. In one embodiment of the invention, the dressing comprises a contact layer and a backing layer, both fabricated from absorbent materials. The contact layer has an inboard surface and an opposing outboard surface defined. The outboard surface of the contact layer is adapted to be applied to a patient's skin surrounding a wound. The contact layer further defines a die-cut through-opening substantially in the center of the contact layer. Adhering to the inboard surface of the contact layer is the backing layer, which likewise has an inboard surface and an opposing outboard surface defined. Medicine is received in the area defined by the through-opening in the contact layer and the inboard surface of the backing layer. A polymer film covers the outboard surface of the backing layer to prevent the escape of medicine from the dressing through the backing layer. A removable release film covers the adhesive-covered outboard surface of the contact layer, the reception area and the medicine until the dressing is ready to be used.

### CROSS-REFERENCE TO RELATED APPLICATIONS

Not Applicable.

### STATEMENT REGARDING FEDERALLY-SPONSORED RESEARCH OR DEVELOPMENT

Not Applicable.

### BACKGROUND OF THE INVENTION

1. Field of Invention

The invention relates to the field of dressings for wounds. Specifically, the invention relates to a laminated and layered dressing that incorporates an adhesive for adhering the dressing to the skin and a space that allows the dressing to hold medicine against the wound.

2. Description of the Related Art

Several competing factors influence the design of a dressing for a wound. A dressing should cover a wound and play its part to prevent infection or the introduction of undesired foreign matter into the wound. It is desirable that a dressing should be designed to stay in place once applied until intentionally removed. At the same time, it is desirable that a dressing should not irritate the wound or surrounding tissue. Additionally, it has been found advantageous in many circumstances to apply some medication or disinfectant, often in a liquid or gel form, to the wound while the dressing is in use. A dressing should also in many circumstances allow the wound to “breathe”—i.e. permit air to permeate the dressing and reach the wound—and allow moisture to enter and escape the wound area, without allowing the escape of medicine or the introduction of contaminants.

In this context, the term “wound” encompasses many kinds of injuries and dermatological phenomena, including traumatic wounds such as minor abrasions and lacerations, surgical wounds and post-operative wounds, burns, punctures and the like.

### BRIEF SUMMARY OF THE INVENTION

Disclosed herein is a laminate dressing defining a reception area adapted to receive and hold medicine. In one embodiment of the invention, the dressing comprises a contact layer and a backing layer, both fabricated from absorbent materials. The contact layer has defined two opposing surfaces: an inboard surface that adheres to the backing layer, and an outboard surface that is coated with adhesive and adapted to be applied to a patient's skin surrounding a wound. The contact layer further defines a die-cut through-opening substantially in the center of the contact layer. Medicine is received in a reception area defined by the through-opening in the contact layer and the inboard surface of the backing layer. When the dressing is used, the reception area and medicine are placed over the wound, while the contact layer with adhesive, bordering the reception area, is applied to the healthy skin surrounding the wound. In this way, the dressing is held in place on the patient's skin without the potentially irritating adhesive coming into contact with the wound. A polymer film covers the outboard surface of the backing layer to prevent the escape of medicine from the dressing through the backing layer.

A second, removable release film covers the adhesive-covered outboard surface of the contact layer, the reception area and the medicine until the dressing is ready to be used. In some embodiments of the invention, the dressing also comprises a tab or folded liner that helps in removing the second polymer film from the dressing before use.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 is a perspective view of one embodiment of the invention;

FIG. 2 is an exploded view of the embodiment shown in FIG. 1;

FIG. 3 is a cross-section of the embodiment shown in FIG. 1;

FIG. 4 is a plan view of one embodiment of the invention with removable release film;

FIG. 5 is a cross-section of the embodiment shown in FIG. 4;

FIG. 6 is a view of the embodiment shown in FIG. 4 as the removable release film is being separated from the dressing;

FIG. 7 is a perspective view of the embodiment shown in FIG. 4 in use on the skin of a patient;

FIG. 8 is a cross-section of the embodiment shown in FIG. 7 in use on the skin of a patient, covering a wound;

FIG. 9 is a plan view of another embodiment of the invention, including a tab or liner;

FIG. 10 is a cross-section of the embodiment shown in FIG. 9; and

FIG. 11 is a view of the embodiment shown in FIG. 9 as the removable release film is being separated from the dressing.

### DETAILED DESCRIPTION OF THE INVENTION

The present invention is described more fully hereinafter. From the outset, it is worth noting that this invention may be embodied in many different forms and should not be construed as limited to the specific embodiments described herein.

The present invention broadly comprises a laminate dressing defining a reception area adapted to receive and hold medicine. In one embodiment of the invention, the dressing comprises a contact layer and a backing layer, both fabricated from an absorbent spun-laced material such as Sontara. The contact layer has an inboard surface and an opposed outboard surface defined. The outboard surface of the contact layer is adapted to be applied to a patient's skin surrounding a wound. The contact layer further defines a through-opening substantially in the center of the contact layer. Adhering to the inboard surface of the contact layer is the backing layer, which likewise has an inboard surface and an opposed outboard surface defined. Medicine is received in the area defined by the through-opening in the contact layer and the inboard surface of the backing layer. A polymer film covers the outboard surface of the backing layer to prevent or limit the escape of medicine from the dressing through the backing layer.

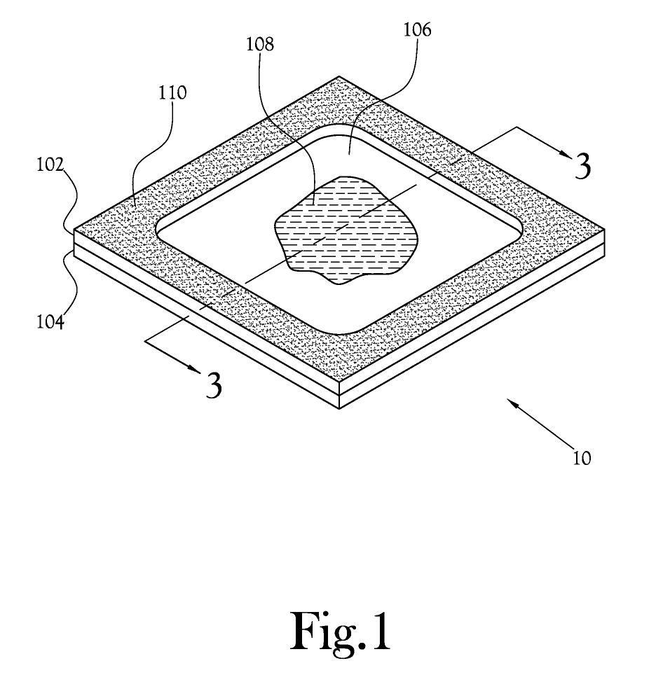

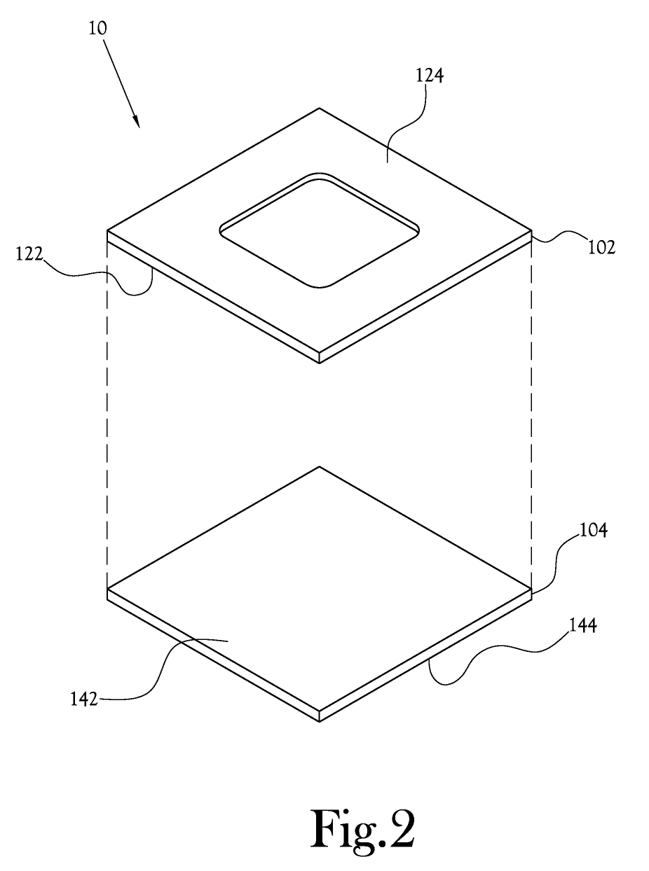

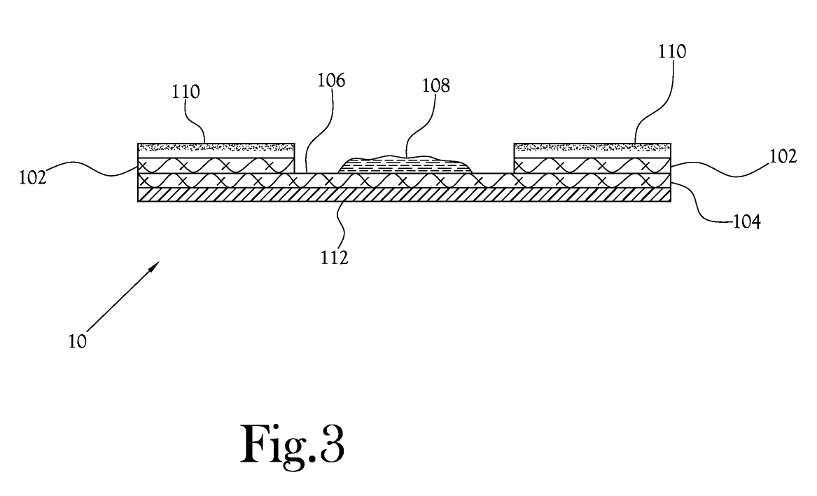

Referring to FIG. 1, one embodiment 10 of the dressing comprises a contact layer 102 and a backing layer 104. Referring to FIG. 2, the contact layer 102 has an inboard surface 122 and an opposed outboard surface 124, and the backing layer 104 has an inboard surface 142 and an opposed outboard surface 144. As shown in FIG. 1 and FIG. 2, the contact layer 102 defines a through-opening, which together with the inboard surface 142 of the backing layer 104 defines a reception area 106 designed to receive medicine 108. The inboard surface 122 of the contact layer 102 adheres to the inboard surface 142 of the backing layer 104. The outboard surface 124 of the contact layer 102 is covered with an adhesive 110, as shown in FIG. 1. When the dressing 10 is applied to a patient's wound, the adhesive 110 allows the outboard surface 124 of the contact layer 102 and hence the entire dressing 10 to adhere to the skin surrounding the wound. At the same time, the reception area 106, which is placed over the wound itself, is free of adhesive. In this way, the dressing 10 adheres to the patient's skin and covers the wound without any of the adhesive coming into contact with the wound. The reception area 106 also allows the medicine 108 to be deposited on the wound. Referring initially to FIG. 3 and again to FIG. 1 and FIG. 2, the medicine 108 rests upon the inboard surface 142 of the backing layer 104 within the reception area 106. Because the backing layer 104 is in many embodiments made from an absorbent material, some amount of the medicine 108 may seep into the backing layer 104. To inhibit the medicine 108 from leaking through the backing layer 104, a polymer film or coating 112 covers the outboard surface 144 of the backing layer 104 opposite the inboard surface 142 upon which the medicine 108 is deposited.

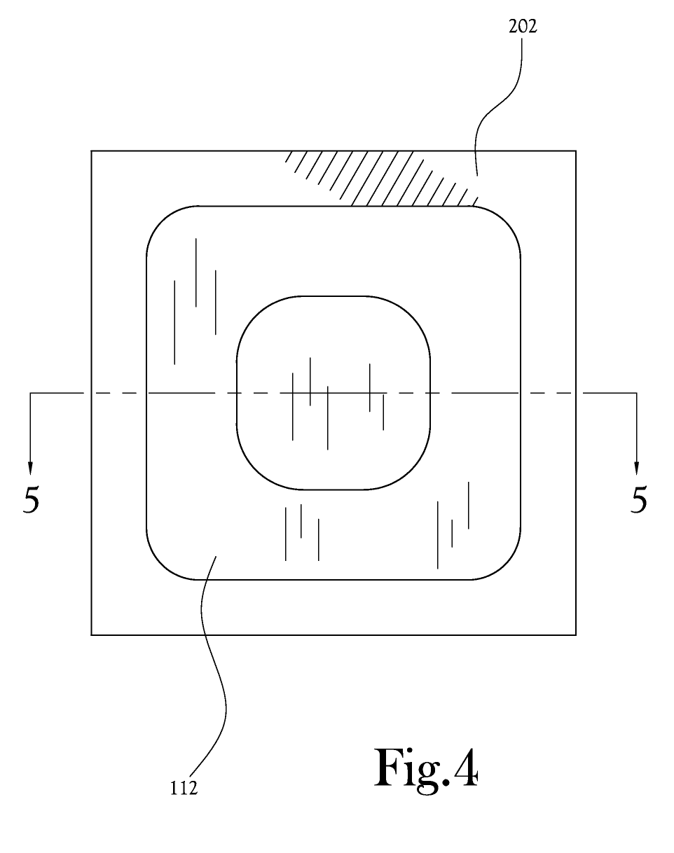

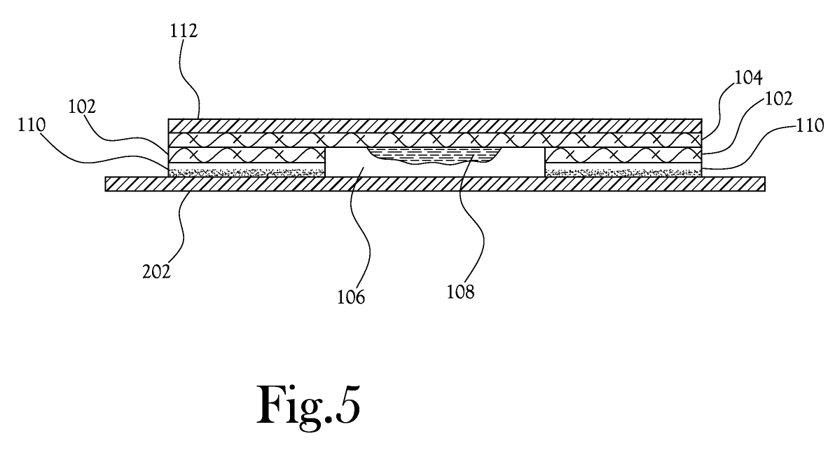

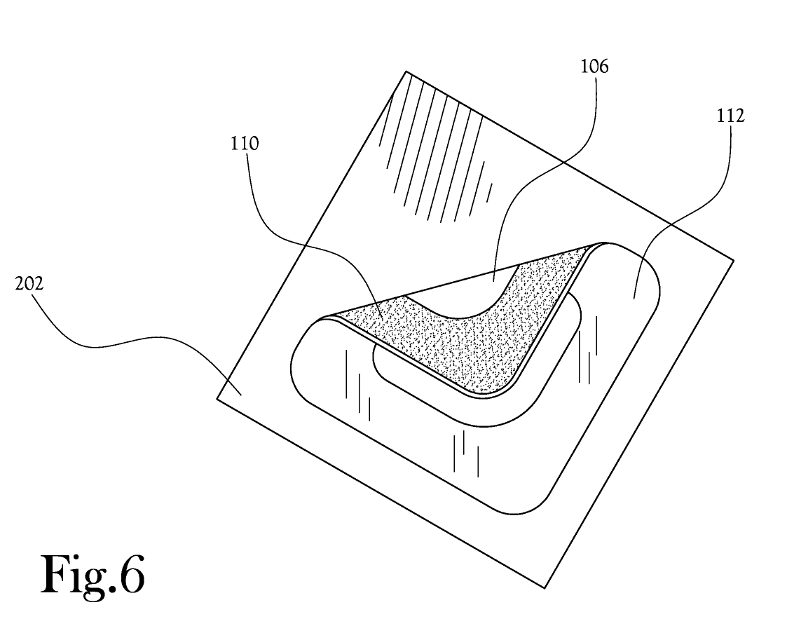

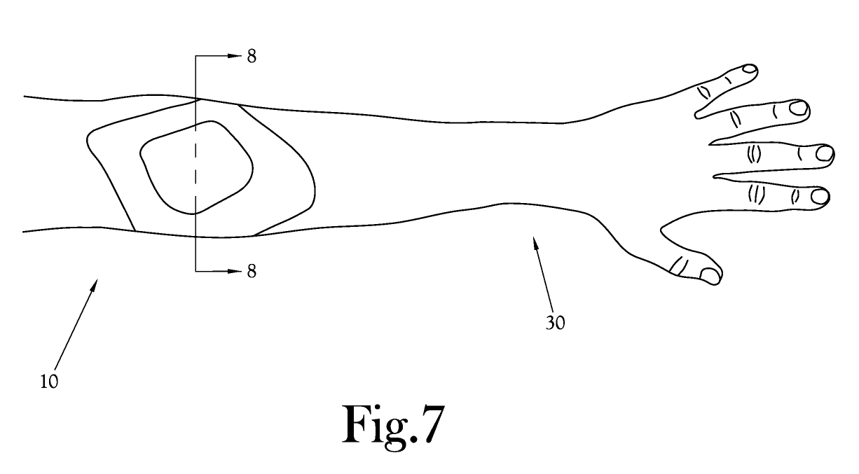

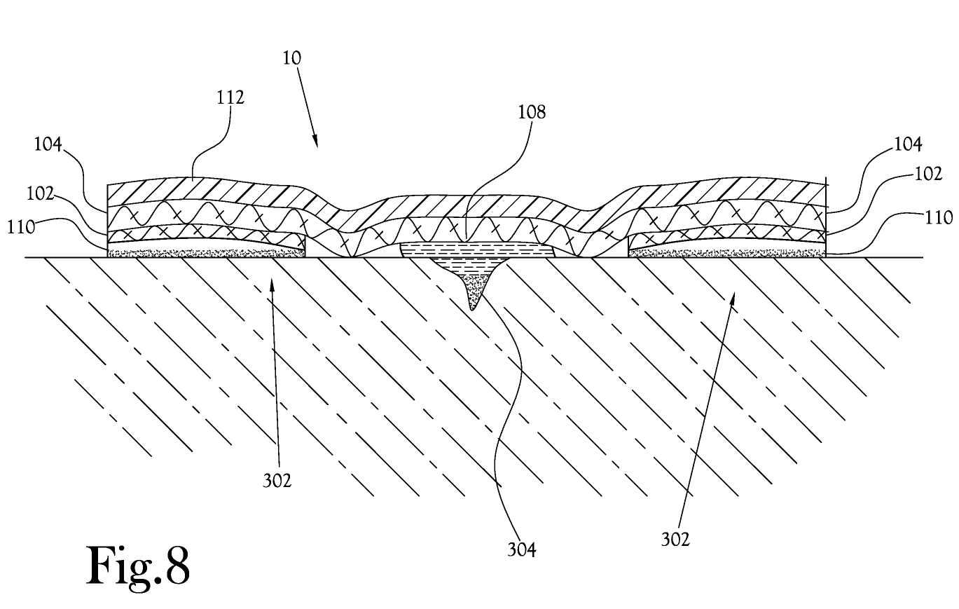

In order to prevent contamination of the dressing 10 and maintain the adhesive power of the adhesive 110 until the dressing is applied to a patient's skin, a removable release film 202 loosely adheres to the outboard surface 124 of the contact layer 102 and covers the reception area 106 and the outboard surface 124 of the contact layer 102 bearing the adhesive 110, as shown in FIG. 4 and in FIG. 5. Referring to FIG. 4, in some embodiments the removable release film 202 has a perimeter larger than the perimeter of the dressing 10. Referring to FIG. 6, when the dressing 10 is ready to be applied to a patient's wound, a corner or edge of the dressing 10 is peeled back from the removable release film 202 to expose the outboard surface 124 of the contact layer 102 with its adhesive 110. After or as the removable release film 202 is separated from the dressing 10, the dressing 10 is applied to the patient 30 as shown in FIG. 7. Referring to FIG. 8, the adhesive 110 on the outboard surface 124 of the contact layer 102 is applied to skin 302 around the wound, while the reception area 106 and the medicine 108 cover the wound itself 304.

As noted above, the contact layer 102 of the dressing 10 is fabricated from a flexible, absorbent material, adapted to carry the adhesive 110 on the outboard surface 124 and to adhere with the inboard surface 122 to the backing layer 104. Likewise, the backing layer 104 of the dressing 10 is fabricated from a flexible, absorbent material which, in some but not all embodiments, is the same material as used for the contact layer 102. The material of the backing layer 104 is also adapted to carry the medicine 108. Further, as some portion of the backing layer 104 comes into contact with the patient's wound 304 when applied, as shown in FIG. 8, the material of the backing layer 104 is also adapted not to irritate the wound 304. A spun-laced fabric, such as Sontara®, is effective for both the contact layer 102 and the backing layer 104, but other materials are also effective and known in the art of dressings and bandages.

The polymer film 112, used to cover the outboard surface 144 of the backing layer 104, in some embodiments comprises polyethylene or polyurethane. The removable release film 202, used to cover the outboard surface 124 of the contact layer 102 before application of the dressing 10 to a patient, in some embodiments comprises polyethylene or polyurethane. Those of skill in the art will recognize that other polymeric materials and other similar materials are effective for each film. In many embodiments, the first polymer film 112 is water-permeable and air-permeable, thus allowing the dressing and wound to breathe while still preventing or limiting the escape of the medicine 108.

In some embodiments, the through-opening in the contact layer 102 is cut from the fabric by a die during the manufacturing process. The contact layer 102 with die-cut through-opening is then adhered to the backing layer 104. The medicine 108 is then deposited in the receiving area 106 before the dressing 10 is covered with the removable release film 202. In many embodiments of the invention, the medicine 108 comprises a gel or liquid. In some embodiments, the medicine 108 comprises petrolatum or Vaseline. In some embodiments, the medicine 108 comprises an antimicrobial agent. Other chemicals and compounds useful for treating the wound and preventing infection of the wound will be apparent to those of skill in the art. In many cases, the medicine 108 to be applied will depend upon the type of wound to be treated.

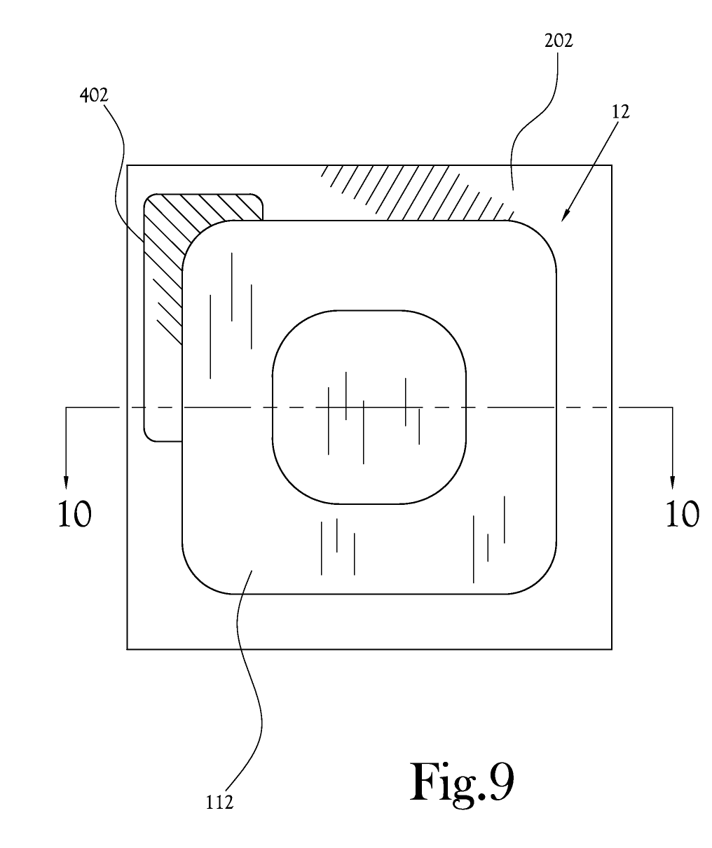

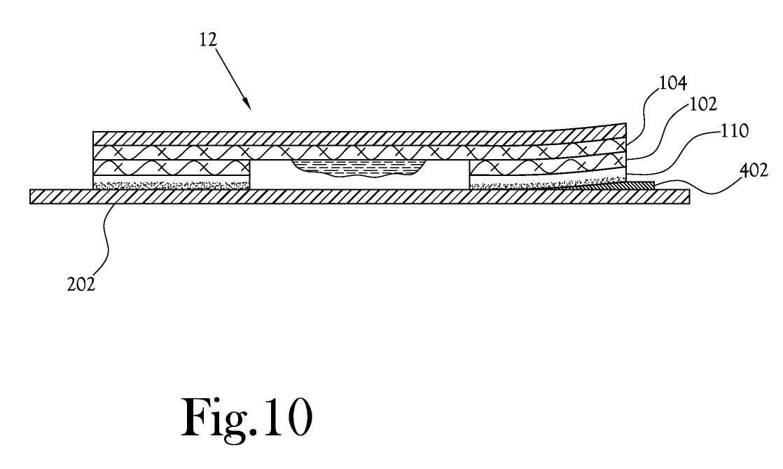

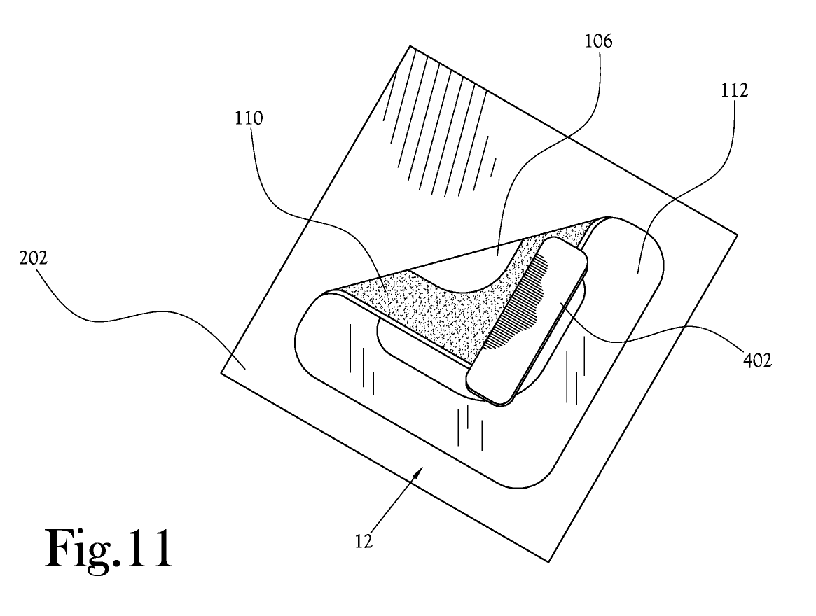

Referring to FIG. 9 and to FIG. 10, an alternative embodiment of the invention 12 comprises a tab or folded liner 402 positioned along at least part of at least one edge of the dressing 12 between the removable release film 202 and the adhesive 110 on the outboard surface 124 of the contact layer 102. As shown in FIG. 11, during the separation of the removable polymer film 202 from the contact layer 102, the tab or liner 402 may be grasped to pull the dressing 12 away from the removable polymer film 202. When the dressing 12 has been applied to the patient's wound, the tab or liner 402 may in turn be pulled away from the dressing, allowing the full adhesive-coated surface of the contact layer 102 to adhere to the patient's skin.

While the dressings shown in the various Figures have generally square shapes, other shapes are possible for the dressing, including but not limited to circular, oval and rectangular dressings.

While the present invention has been illustrated by description of several embodiments and while the illustrative embodiments have been described in detail, it is not the intention of the applicant to restrict or in any way limit the scope of the appended claims to such detail. Additional modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

Having thus described the aforementioned invention, what is claimed is:

1. A dressing for a wound comprising a laminate including
a contact layer including an absorbent material, said contact layer defining an inboard surface and an opposing outboard surface, said contact layer defining a through-opening substantially centrally located thereon;
an adhesive applied to said outboard surface of said contact layer, said adhesive being adapted to securing the dressing to human skin;
a backing layer including an absorbent material and defining an inboard surface and an opposing outboard surface, said outboard surface being substantially covered by a moisture-permeable polymer coating, said inboard surface of said backing layer being adhered to said inboard surface of said contact layer, said inboard surface of said backing layer and said through-opening defined by said contact layer cooperating to form a reception area adapted to receive a quantity of medication; and
a release film covering said adhesive and said outboard surface of said contact layer, said release film loosely adhering to said outboard surface of said contact layer, whereby said release film protects said adhesive until the dressing is ready to be applied to a wound.

2. The dressing of claim 1 further comprising a tab disposed between a portion of said outboard surface of said contact layer and a portion of said release film, said tab positioned near at least one edge of said contact layer and at least one edge of said release film, said tab loosely held by said adhesive on said outboard surface of said contact layer, said tab including a portion extending away from said at least one edge of said contact layer and adapted to being grasped, whereby said portion of said tab may be grasped to assist in the separation of said contact layer from said release film before application of the dressing to a wound.

3. The dressing of claim 1 further comprising a second adhesive disposed between said inboard surface of said contact layer and said inboard surface of said backing layer.

4. A dressing for a wound, said dressing comprising:
a first layer adapted to be placed over the wound, said first layer fabricated from an absorbent material, said first layer defining a top surface and a bottom surface, said bottom surface defining a contact area and a reception area, said contact area having a greater thickness than said reception area, said contact area surrounding said reception area, said reception area located substantially centrally on said bottom surface, said reception area adapted to receive a quantity of medication therein;
a second layer adhered to said top surface of said first layer, said second layer fabricated from a substantially water-permeable and air-permeable composition that is substantially impermeable with respect to the medication, whereby said second layer is adapted to allow the wound to breathe while preventing leakage of the medication from said top surface of said first layer;
an adhesive adhered to said contact area of said bottom surface of said first layer, said adhesive adapted to adhere said contact surface to the skin around the wound; and
a release film in contact with and covering said adhesive, said release film loosely held by said adhesive, said release film adapted to be separated from said adhesive, whereby said film protects said adhesive until the dressing is ready for use.

5. The dressing of claim 4 wherein said first layer comprises a top sublayer and a bottom sublayer, said bottom sublayer defining a through-opening substantially centrally located thereon, said through-opening and said top sublayer cooperating to form said reception area.

6. The dressing of claim 5 wherein said top sublayer is adhered to said bottom sublayer.

7. The dressing of claim 4 wherein said second layer is a polymeric film adhered to said top surface of said first layer.

8. The dressing of claim 4 wherein said second layer is a polymeric composition applied and bonding with said top surface of said first layer.

9. The dressing of claim 4 further comprising a folded liner disposed between a portion of said bottom surface of said first layer and a portion of said film, said liner being positioned near at least one edge of said first layer and at least one edge of said film, said liner loosely held by said adhesive on said bottom surface of said first layer, said liner including a portion extending away from said at least one edge of said first layer and adapted to being grasped, whereby said portion of said liner may be grasped to assist in the separation of said first layer from said film before application of the dressing to a wound.

10. A method for making a dressing comprising the steps of
taking a first portion fashioned of an absorbent material and cutting a through-passage in said portion with a die,
taking a second portion fashioned of an absorbent material, said second portion having a perimeter at least larger than the perimeter of said through-passage in said first portion,
adhering a first surface of said first portion to a first surface of said second portion, whereby part of said second portion covers said through-passage in said first portion,
coating a second surface of said second portion in a water-permeable polymer,
depositing some quantity of medication on said first surface of said second portion on said part of said second portion covering said through-passage in said first portion,
coating a second surface of said first portion in an adhesive adapted to affix said dressing to human skin, and
covering said second surface of said first portion, said through-passage in said first portion, and said quantity of medication with a polymer film adapted to lightly adhere to said second surface of said first portion, said polymer film further adapted to be removed from said second surface of said first portion before said dressing is to be applied to a wound.
